(19) United States
(12) Patent Application Publication
(10) Pub. No.: US 2025/0265816 A1
(43) Pub. Date: Aug. 21, 2025

(54) SYSTEM, NON-TRANSITORY STORAGE MEDIUM AND ELECTRONIC DEVICE FOR RECOGNIZING AUTISM BASED ON HYBRID DEEP LEARNING

(71) Applicant: Shandong University, Jinan (CN)

(72) Inventors: Xin MA, Jinan (CN); Xiang LI, Jinan (CN)

(21) Appl. No.: 19/072,928

(22) Filed: Mar. 6, 2025

(30) Foreign Application Priority Data
Mar. 7, 2024 (CN) 202410260848.1

(51) Int. Cl. G06V 10/764; A61B 5/00; A61B 5/11; G06V 10/25; G06V 10/34; G06V 10/766; G06V 10/77; G06V 10/80; G06V 10/82; G06V 20/40; G06V 40/16; G06V 40/20; G16H 50/20

(52) U.S. Cl. CPC G06V 10/764; A61B 5/1124; A61B 5/4076; G06V 10/25; G06V 10/34; G06V 10/766; G06V 10/7715; G06V 10/806; G06V 10/82; G06V 20/41; G06V 20/46; G06V 20/49; G06V 40/174; G06V 40/20; G16H 50/20; A61B 2503/06; G06V 2201/03

(57) ABSTRACT

A system for recognizing autism based on hybrid deep learning includes a data acquisition module, a skeleton keypoint extraction module and a recognition and classification module. The data acquisition module is configured for obtaining a dataset based on a parent-child dyad block game protocol. The skeleton keypoint extraction module is configured for identifying a plurality of skeleton keypoints of a target and a position of each of the plurality of skeleton keypoints in the video data based on a high-resolution network to generate a skeleton sequence. The recognition and classification module is configured for classifying the child into autism spectrum disorder (ASD) children and typically developing (TD) children by inputting the skeleton sequence in a graph form into a Two-stream Graph Attention Long Short-Term Memory (2sG-ALSTM) network architecture.

### CROSS-REFERENCE TO RELATED APPLICATIONS

This application claims the benefit of priority from Chinese Patent Application No. 202410260848.1, filed on Mar. 7, 2024. The content of the aforementioned application, including any intervening amendments made thereto, is incorporated herein by reference in its entirety.

### TECHNICAL FIELD

This application relates to computer-aided recognition, and more particularly to a system, non-transitory storage medium and electronic device for recognizing autism based on hybrid deep learning.

### BACKGROUND

This section is provided solely to offer background technical information related to this disclosure and does not necessarily constitute prior art.

Autism Spectrum Disorder (ASD) is a rapidly growing neurodevelopmental disorder that primarily manifests in early childhood. Timely intervention is crucial for the growth and development of children with ASD, yet traditional auxiliary clinical screening methods are time-consuming and lack measurable indicators.

Computer vision (CV) technology is increasingly being used to analyze and recognize human behaviors, offering a more objective and efficient means of ASD detection. Researchers have developed various protocols, such as the name-calling response, expressing needs by the index finger pointing (ENIFP) and the robot-assisted protocol (RAP), to analyze key behaviors including head, finger, and facial movements. These protocols help assess the quality of joint attention and social communication, enhanced by CV capabilities. In addition, experiments have utilized biomarkers like eye tracking, head movement, and motion to identify ASD characteristics.

However, protocols that focus on a single biomarker may not fully capture the complexity of social interactions and cognitive behaviors. Traditional scales such as Autism Diagnostic Interview-Revised (ADI-R), Autism Screening Instrument for Educational Planning-Third Edition (ASIEP-3) or Screening Tool for Autism in Toddlers (STAT), although typically used to evaluate ASD symptoms, require lengthy direct observation by clinicians. Although deep learning has become a key tool in enhancing ASD screening, aiming to surpass traditional performance benchmarks, there is a need for improvement in the temporal dynamic modeling of complex actions.

Existing 3D Convolutional Neural Network (3DCNN) cannot achieve higher performance, which can be attributed to the inherent characteristics of the activities in the collected dataset. Unlike behavioral datasets, it is difficult for CNN to extract useful information from the background to accurately recognize behavioral activities.

### SUMMARY

This disclosure proposes a system for recognizing autism based on hybrid deep learning and introduces a Parent-Child Dyad Grouping Game (PCB) protocol to construct a dataset. PCB protocol is designed to capture ASD-related behaviors specific to young children, providing standardized dataset guidance for future consistent assessments. The comprehensive annotated PCB video dataset is more extensive than previous datasets in terms of the number of participants and the duration of individual sessions. This dataset records children's interactive behaviors, serving as a valuable resource for fine-grained behavioral analysis in early ASD screening.

According to some embodiments, the present disclosure adopts the following technical solutions.

A system for recognizing autism based on hybrid deep learning, comprising:
a data acquisition module;
a skeleton keypoint extraction module; and
a recognition and classification module;
wherein the data acquisition module is configured for obtaining a dataset based on a parent-child dyad block game protocol through steps of:
capturing, by a camera, a facial expression and a body movement of child and a parent when the child and the parent perform a task sequence to obtain a plurality of video clips; and
organizing the plurality of video clips as the dataset;
wherein the dataset is a video data; the video data is continuous; the parent-child dyad block game protocol is the task sequence;
the skeleton keypoint extraction module is configured for identifying a plurality of skeleton keypoints of a target and a position of each of the plurality of skeleton keypoints in the video data based on a high-resolution network to generate a skeleton sequence; wherein the target comprises the child and the parent; the skeleton sequence comprises a coordinate of each of the plurality of skeleton keypoints;
the recognition and classification module is configured for classifying the child into autism spectrum disorder (ASD) children and typically developing (TD) children by inputting the skeleton sequence in a graph form into a Two-stream Graph Attention Long Short-Term Memory (2sG-ALSTM) network architecture to through steps of:
classifying a skeleton data in the skeleton sequence into a data of an upper body of the target and a data of a head of the target; wherein the 2sG-ALSTM network architecture is a human skeleton action recognition method based on a graph convolutional network (GCN) and a long short-term memory (LSTM) network;
representing the data of the upper body as a first graph with self-loop and the data of the head as a second graph with self-loop;
transforming the first graph into a first adjacency matrix set and the second graph into a second adjacency matrix set;
selecting a first matrix from the first adjacency matrix set based on a multi-scale spatial partitioning strategy; selecting a second matrix from the second adjacency matrix set based on a neighbor set partitioning strategy;
mapping the first matrix from a posture space to a feature space to obtain a first vector and mapping the second matrix from the posture space to the feature space to obtain a second vector;
extracting a first posture sequence feature related to a movement of the upper body from the first vector and a second posture sequence feature related to a movement of the head from the second vector;
fusing the first posture sequence feature and second posture sequence feature to obtain a first comprehensive posture sequence feature; and inputting the first comprehensive posture sequence feature into the LSTM network; and
automatically assigning an attention weight to each frame within the first comprehensive posture sequence feature through a temporal attention module of the LSTM network to obtain a second comprehensive posture sequence feature;
classifying the child into the ASD children and the TD children based on the second comprehensive posture sequence feature;
wherein a route is formed by connecting the plurality of skeleton keypoints; a route distance is a distance between two of the plurality of skeleton keypoints in the route; a first adjacency matrix in the first adjacency matrix set represents a neighbor relationship between skeleton keypoints of the upper body among the plurality of skeleton keypoints corresponding to the route distance; step of selecting the first matrix from the first adjacency matrix set based on the multi-scale spatial partitioning strategy comprises:
setting a K value;
selecting the first matrix corresponding to a route distance less or equal to the K value from the first adjacency matrix set; and
wherein a second adjacency matrix in the second adjacency matrix set represents a neighbor relationship between a root skeleton keypoint and a non-root skeleton keypoint corresponding to the route distance; the root skeleton keypoint and the non-root skeleton keypoint belong to skeleton keypoints of the head among the plurality of skeleton keypoints; step of selecting the second matrix from the second adjacency matrix set based on the neighbor set partitioning strategy comprises:
setting a Q value and one of the skeleton keypoints of the head as the root skeleton keypoint; and
selecting the second matrix corresponding to the route distance less or equal to the Q value from the second adjacency matrix set.

In an embodiment, the skeleton keypoint extraction module is configured for identifying the target through steps of:
segmenting the video data into frame images;
inputting the frame images into a Faster Region-based Convolutional Neural Network (R-CNN);
extracting feature images from the frame images through a backbone network of the R-CNN;
generating human candidate regions based on the feature images through a region proposal network (RPN) of the R-CNN; and
performing classification and bounding box regression for the human candidate regions through a detection network of the R-CNN to convert the human candidate regions with varying sizes into a feature vector with fixed-size to output a coordinate of a bounding box of the target, a type of the target and a prediction probability.

In an embodiment, the skeleton keypoint extraction module is configured for obtaining the skeleton sequence through steps of:
inputting the coordinate of the bounding box of the target, the type of the target prediction and the prediction probability into a High-Resolution Network (HRNet); wherein the HRNet comprises a plurality of parallel branches;
extracting space feature from the human candidate regions through the plurality of parallel branches with varying sizes of convolution kernels and varying strides to obtain multi-scale feature images; and
fusing the multi-scale feature images at both a pixel level and a channel level through a fully connected layer of the HRNet to obtain the coordinate of each of the plurality of skeleton keypoints and a confidence level of each of the plurality of skeleton keypoints to obtain the skeleton sequence.

In an embodiment, the first graph and the second graph are represented as G={N,E}; N represents a set of the plurality of skeleton keypoints; E represents lines connecting the plurality of skeleton keypoints.

In an embodiment, the GCN comprises GCN block groups; each of GCN block groups comprises three GCN blocks; the GCN block groups are connected in series; a first residual connection is set in each of the GCN block groups; a second residual connection is set between an input of a first GCN block group and an output of a last GCN block group; the recognition and classification module is configured for classifying the child into the ASD children and the TD children through steps of:
mapping the first matrix from the posture space to the feature space to obtain the first vector and mapping the second matrix from the posture space to the feature space to obtain the second vector through a first GCN block of the first block group;
extracting the first posture sequence feature from the first vector and the second posture sequence feature from the second vector by learning residuals generated by the first residual connection and the second residual connection;
performing adaptive fusion for the first posture sequence feature and the second posture sequence feature to obtain the first comprehensive posture sequence feature;
and inputting the first comprehensive posture sequence feature into the LSTM network; and
assigning the attention weight to each frame within the first comprehensive posture sequence feature through the temporal attention module of the LSTM network to obtain the second comprehensive posture sequence feature; and
predicting a probability based on the second comprehensive posture sequence feature through a softmax algorithm to classify the child into the ASD children and the TD children.

A non-transitory storage medium, wherein the non-transitory storage medium stores a computer program; and the computer program is configured to be executed by a processor to implement steps of:
obtaining a dataset based on a parent-child dyad block game protocol through steps of:
   capturing, by a camera, a facial expression and a body movement of a child and a parent when the child and the parent perform a task sequence to obtain a plurality of video clips; and
   organizing the plurality of video clips as the dataset;
   wherein the dataset is a video data; the video data is continuous; the parent-child dyad block game protocol is the task sequence;
identifying a plurality of skeleton keypoints of a target and a position of each of the plurality of skeleton keypoints in the video data based on a high-resolution network to generate a skeleton sequence; wherein the target comprises the child and the parent;
classifying the child into autism spectrum disorder (ASD) children and typically developing (TD) children by inputting the skeleton sequence in a graph form into a Two-stream Graph Attention Long Short-Term Memory (2sG-ALSTM) network architecture to through steps of:
   classifying a skeleton data in the skeleton sequence into a data of an upper body of the target and a data of a head of the target; wherein the 2sG-ALSTM network architecture is a human skeleton action recognition method based on a graph convolutional network (GCN) and a long short-term memory (LSTM) network;
   representing the data of the upper body as a first graph with self-loop and the data of the head as a second graph with self-loop;
   transforming the first graph into a first adjacency matrix set and the second graph into a second adjacency matrix set;
   selecting a first matrix from the first adjacency matrix set based on a multi-scale spatial partitioning strategy; selecting a second matrix from the second adjacency matrix set based on a neighbor set partitioning strategy;
   mapping the first matrix from a posture space to a feature space to obtain a first vector and mapping the second matrix from the posture space to the feature space to obtain a second vector;
   extracting a first posture sequence feature related to a movement of the upper body from the first vector and a second posture sequence feature related to a movement of the head from the second vector;
   fusing the first posture sequence feature and second posture sequence feature to obtain a first comprehensive posture sequence feature; and inputting the first comprehensive posture sequence feature into the LSTM network; and
   automatically assigning an attention weight to each frame within the first comprehensive posture sequence feature through a temporal attention module of the LSTM network to obtain a second comprehensive posture sequence feature;
   classifying the child into the ASD children and the TD children based on the second comprehensive posture sequence feature;
wherein a route is formed by connecting the plurality of skeleton keypoints; a route distance is a distance between two of the plurality of skeleton keypoints in the route; a first adjacency matrix in the first adjacency matrix set represents a neighbor relationship between skeleton keypoints of the upper body among the plurality of skeleton keypoints corresponding to the route distance; step of selecting the first matrix from the first adjacency matrix set based on the multi-scale spatial partitioning strategy comprises:
   setting a first value;
   selecting the first matrix corresponding to a route distance less or equal to the first value from the first adjacency matrix set; and
wherein a second adjacency matrix in the second adjacency matrix set represents a neighbor relationship between a root skeleton keypoint and a non-root skeleton keypoint corresponding to the route distance; the root skeleton keypoint and the non-root skeleton keypoint belong to skeleton keypoints of the head among the plurality of skeleton keypoints; step of selecting the second matrix from the second adjacency matrix set based on the neighbor set partitioning strategy comprises:
   setting a second value and one of the skeleton keypoints of the head as the root skeleton keypoint; and
   selecting the second matrix corresponding to the route distance less or equal to the second value from the second adjacency matrix set.

An electronic device, comprising:
a processor;
a memory; and
a program;
wherein the program is stored on the memory; and the processor is configured to execute the program to implement steps of:
obtaining a dataset based on a parent-child dyad block game protocol through steps of:
   capturing, by a camera, a facial expression and a body movement of a child and a parent when the child and the parent perform a task sequence to obtain a plurality of video clips; and
   organizing the plurality of video clips as the dataset;
   wherein the dataset is a video data; the video data is continuous; the parent-child dyad block game protocol is the task sequence;
identifying a plurality of skeleton keypoints of a target and a position of each of the plurality of skeleton keypoints in the video data based on a high-resolution network to generate a skeleton sequence; wherein the target comprises the children and the parent;
classifying the child into autism spectrum disorder (ASD) children and typically developing (TD) children by inputting the skeleton sequence in a graph form into a Two-stream Graph Attention Long Short-Term Memory (2sG-ALSTM) network architecture to through steps of:
   classifying a skeleton data in the skeleton sequence into a data of an upper body of the target and a data of a head of the target; wherein the 2sG-ALSTM network architecture is a human skeleton action recognition method based on a graph convolutional network (GCN) and a long short-term memory (LSTM) network;
   representing the data of the upper body as a first graph with self-loop and the data of the head as a second graph with self-loop;
   transforming the first graph into a first adjacency matrix set and the second graph into a second adjacency matrix set;
   selecting a first matrix from the first adjacency matrix set based on a multi-scale spatial partitioning strategy; selecting a second matrix from the second adjacency matrix set based on a neighbor set partitioning strategy;
   mapping the first matrix from a posture space to a feature space to obtain a first vector and mapping the second matrix from the posture space to the feature space to obtain a second vector;
   extracting a first posture sequence feature related to a movement of the upper body from the first vector and a second posture sequence feature related to a movement of the head from the second vector;
   fusing the first posture sequence feature and second posture sequence feature to obtain a first comprehensive posture sequence feature; and inputting the first comprehensive posture sequence feature into the LSTM network; and
   automatically assigning an attention weight to each frame within the first comprehensive posture sequence feature through a temporal attention module of the LSTM network to obtain a second comprehensive posture sequence feature;
   classifying the child into the ASD children and the TD children based on the second comprehensive posture sequence feature;
wherein a route is formed by connecting the plurality of skeleton keypoints; a route distance is a distance between two of the plurality of skeleton keypoints in the route; a first adjacency matrix in the first adjacency matrix set represents a neighbor relationship between skeleton keypoints of the upper body among the plurality of skeleton keypoints corresponding to the route distance; step of selecting the first matrix from the first adjacency matrix set based on the multi-scale spatial partitioning strategy comprises:
   setting a first value;
   selecting the first matrix corresponding to a route distance less or equal to the first value from the first adjacency matrix set; and
wherein a second adjacency matrix in the second adjacency matrix set represents a neighbor relationship between a root skeleton keypoint and a non-root skeleton keypoint corresponding to the route distance; the root skeleton keypoint and the non-root skeleton keypoint belong to skeleton keypoints of the head among the plurality of skeleton keypoints; step of selecting the second matrix from the second adjacency matrix set based on the neighbor set partitioning strategy comprises:
   setting a second value and one of the skeleton keypoints of the head as the root skeleton keypoint; and
selecting the second matrix corresponding to the route distance less or equal to the second value from the second adjacency matrix set.

This disclosure proposes a hybrid deep learning framework for video-based skeletal behavior analysis, 2sG-ALSTM. This framework combines two-stream graph convolution with attention-enhanced LSTM to extract spatiotemporal features from long-term behaviors, demonstrating superior performance in action recognition, robustness, and spatiotemporal feature extraction. The attention layer computes attention weights based on input values, enabling the model to flexibly focus on variations in input data. This approach helps the model capture key information in input sequences more precisely, thereby improving performance. The PCB dataset is utilized to enhance the screening and classification process for ASD in young children.

Compared with traditional methods, this approach achieves higher performance. Integrating 2sG-ALSTM technology, it effectively preserves the spatiotemporal features in the dataset. Simulations further validate this through comparisons between two skeletal-based methods, showing that it achieves relatively higher accuracy compared to LSTM-based skeletal methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings are provided for further understanding of the present disclosure. The illustrative embodiments and their descriptions are intended to explain the disclosure, instead of limiting the scope of the disclosure.

FIG. 1 shows an example frame of the experimental scenario in the collected dataset according to an embodiment of the present disclosure;

FIG. 2 is a flowchart of the stages of the parent-child interaction using props according to an embodiment of the present disclosure;

FIG. 3 schematically shows the 2sG-ALSTM network architecture according to an embodiment of the present disclosure;

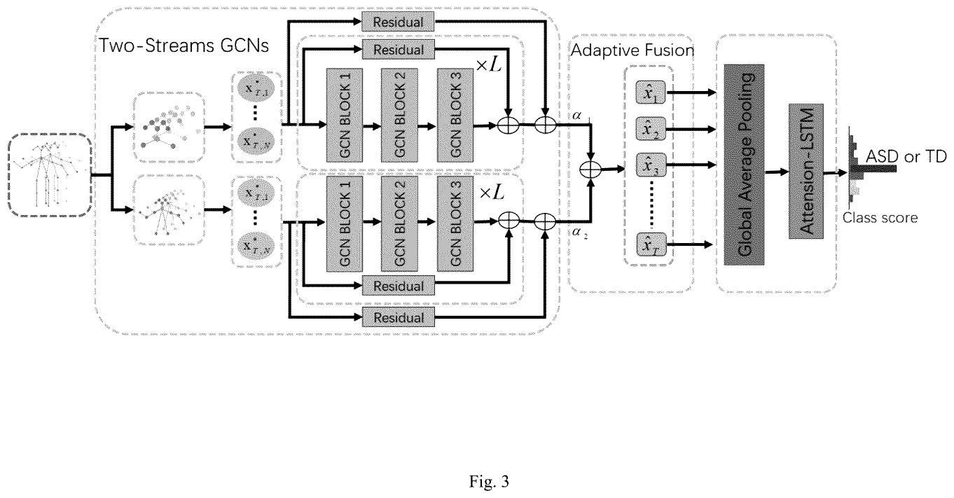

FIG. 4 is a schematic diagram of different neighborhoods of a skeleton keypoint according to an embodiment of the present disclosure;

FIG. 5 schematically shows GCN network block structure according to an embodiment of the present disclosure;

FIG. 6 schematically shows the existing LSTM network structure;

FIG. 7 is a model diagram of the LSTM with an attention mechanism according to an embodiment of the present disclosure;

FIG. 8 schematically shows an example framework of the experimental scenario for ASD children according to an embodiment of the present disclosure; and

FIG. 9 schematically shows an example framework of the experimental scenario for TD children according to an embodiment of the present disclosure.

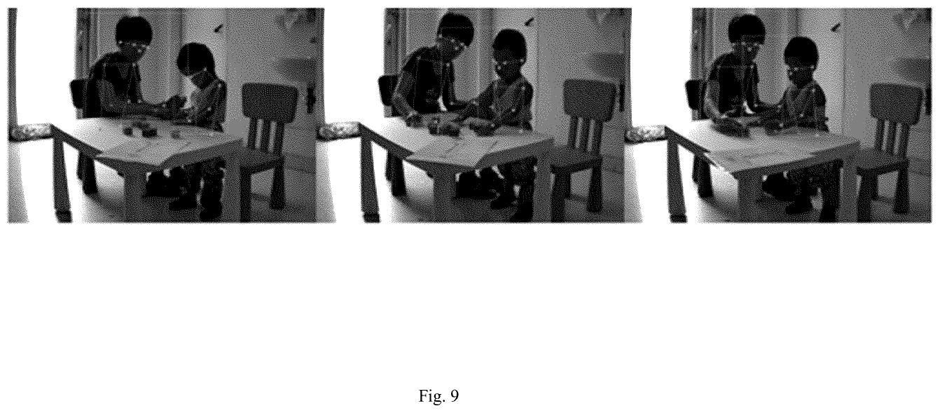

### DETAILED DESCRIPTION OF EMBODIMENTS

The present disclosure will be described in further detail below with reference to the accompanying drawings and embodiments.

It should be noted that the disclosed embodiments are merely exemplary, and intends for further description of the present disclosure. Unless otherwise defined, all technical and scientific terms herein have the same meaning as commonly understood by those skilled in the art to which the present disclosure pertains.

It should be understood that the terminology used in this specification is illustrative for the description of particular embodiments only, and is not intended to limit the disclosure. As used herein, unless the context clearly indicates otherwise, the singular form is also intended to include the plural form. Furthermore, it should be understood that when the terms “comprise” and/or “include” are used in this specification, they indicate the presence of features, steps, operations, devices, components, and/or combinations thereof.

### Embodiment 1

A system for recognizing autism based on hybrid deep learning includes a data acquisition module, a skeleton keypoint extraction module and a recognition and classification module.

The data acquisition module is configured for obtaining a dataset based on a parent-child dyad block game protocol through steps of:

A camera captures a facial expression and a body movement of children and a parent when the children and the parent perform a task sequence to obtain a plurality of video clips. The plurality of video clips are organized as the dataset. The dataset is a video data. The video data is continuous. The parent-child dyad block game protocol is the task sequence.

Specifically, a new PCB protocol based on kinematics and neuroscience research is proposed to identify and differentiate the behavioral patterns of ASD children from typical developing (TD) children. In the context of PCB, a number of video dataset was compiled, which includes block game interactions between 40 children diagnosed with ASD and 89 typically developing children with their parents.

Based on the PCB protocol and video-based behavioral recognition, the interaction between the children and the environment is evaluated, with a particular focus on the movements of the head, hands, and blocks in a structured environment. The PCB protocol is a structured task sequence designed to assess and engage children, especially ASD children, in a controlled experimental setting. A standardized environment is established to enable a consistent evaluation of social attention and cognitive abilities through games. The scene setup and interaction architecture are as follows.

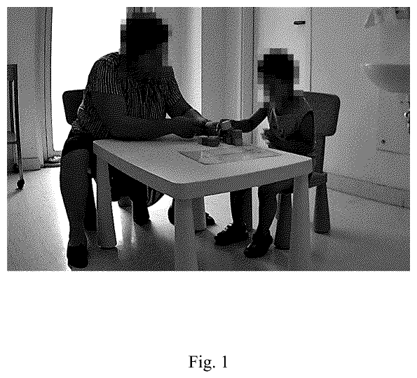

As shown in FIG. 1, an observation platform is designed for a parent-child dyad experimental observation room with an area of approximately 10-15 m², equipped with an appropriate table and two chairs. The experimental materials consist of 10 cubic bricks and a box of irregular bricks designed for children. Various block games are designed for children of various age groups. Before the dyad experiment begins, props and corresponding instructions from the task manual are provided to the children to assist both parents and children in conducting the experiment. In the observation room, a standard RGB camera with recording capabilities is installed to capture the facial expressions and body movements of both the children and the parents. By collecting these data, the behaviors of both parents and children can be analyzed, thereby evaluating the quality of the interactive activities.

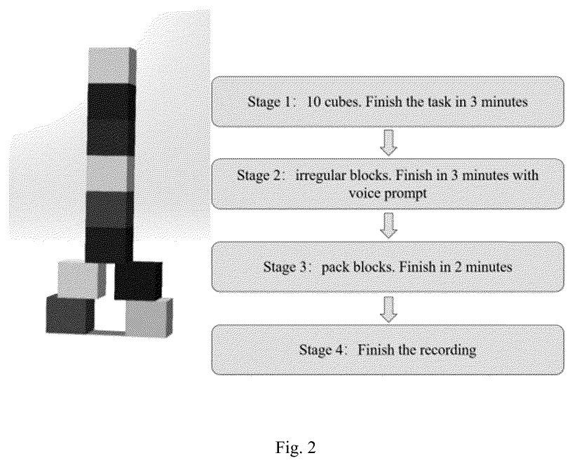

By completing the tasks, including setting specific tasks that require cooperation between the children and the parent, the process of the parent-child dyad is observed. The objective of these tasks is to evaluate the interaction during task completion. The observation process is carried out in three sessions, each lasting approximately 8 minutes, as shown in FIG. 2. The PCB protocol is divided into four distinct stages.

(Stage 1) The children and parent are provided with ten cubes and an instruction manual, and they complete a set task in about three minutes. When time is up, the observer prompts the participants to finish the task. Completing the task within the specified time is not mandatory.

(Stage 2) The observer retrieves the cubes and instruction manual from the participants and gives the children a box of irregular bricks, allowing the children to play freely for about three minutes. The session begins with a voice prompt from the observer.

(Stage 3) The participants are required to spend about two minutes for packing and organizing the bricks used earlier. The session begins after an audio prompt from the observer.

(Stage 4) This stage involves ending the recording process.

The data is then collected into the Parent-Child Block Game (PCB4ASD-ED) dataset, which consists of 187 videos, including 97 ASD video segments and 90 TD video segments, each approximately 20 seconds long. This dataset is nearly twice the size of the previous benchmark dataset SSBD (which had 68 videos), and there are no shared videos between the two datasets.

Since the PCB4ASD-ED dataset includes continuous, long-term video behavioral data, each segment in the videos needs to be manually labeled. The camera parameters used for data collection are fixed, and the entire dataset is converted to a rate of 17 frames per second. After conversion, the dataset contains a total of 72,418 frames. This dataset focuses on the parent-child dyad theme in the block game and is collected from different individuals. However, there are also videos from various environments related to the same theme. In total, there are 129 participants, including 40 ASD children and 89 typically developing children.

Furthermore, Gaussian smoothing is applied to the video frames in the collected dataset to reduce the details in the visual appearance of the subjects. By assigning more weight to the central pixels and less weight to the surrounding pixels, the image is blurred, noise is reduced, and target recognition is improved.

The skeleton keypoint extraction module is configured for identifying a plurality of skeleton keypoints of a target and a position of each of the plurality of skeleton keypoints in the video data based on a high-resolution network to generate a skeleton sequence.

First, since the video data includes interactions between people and objects, it is necessary to detect the subject performing the action. For this purpose, a Faster R-CNN network is employed for child detection. The Faster R-CNN network is an object detection model with 13 convolutional layers, 13 ReLU layers, and 4 pooling layers to detect persons in each frame. The input image is processed through a backbone network to extract feature images, and then the Region Proposal Network (RPN) uses these feature images to generate candidate regions. Finally, the candidate regions are classified and refined with bounding box regression via the detection network. The detection network employs an Rol pooling layer to convert candidate regions varying in sizes into fixed-size feature vectors. The output includes the coordinates of the bounding boxes, the target and their possibilities.

Then, the output from Faster R-CNN is fed into High-Resolution Network (HRNet), which predicts the positions of each skeleton keypoint for the identified human target, thereby performing skeleton keypoint extraction. HRNet contains multiple parallel branches, each using convolution kernel with varying sizes and strides to extract features at varying scales to generate multi-scale feature images. These multi-scale feature images are then fused at both pixel and channel levels to obtain richer and more accurate feature representations. Finally, a fully connected layer with multiple output nodes retrieves the coordinates and confidence scores for each corresponding skeleton keypoint. Both models were pre-trained on the COCO dataset, which has 80 classes. The network takes a tensor of size 1333×800 as an input and outputs a tensor of size 17×3, forming the skeleton sequence.

The recognition and classification module is configured to input the skeleton sequence in a graph form into the 2sG-ALSTM network architecture. First, based on the partition strategy, the skeleton data in the original skeleton sequence is classified into upper body data and head data. The adjacency matrix of the graph with self-loops is divided into multiple matrices. Then, the data is mapped from the pose space to the feature space, where pose sequence features related to the upper body and head movements are extracted. These pose sequence features are input into the LSTM network. In the time-attention module of the LSTM network, specific attention weights are automatically assigned to specific frames in the pose sequence features, and the final classification result is output.

Specifically, this disclosure proposes the 2sG-ALSTM network architecture, a method for recognizing human skeleton action based on dual-stream graph convolution (GCN) and LSTM. Feature extraction is performed based on GCN. First, the normalized skeleton sequence x can be represented as a graph (G={N,E}), where Nis the set of skeleton keypoints; N=[n₁, n₂, . . . , nₖ]; k is the number of keypoints across T frames, and E represents the lines connecting the keypoints. The intra-frame lines are defined based on the natural connections between keypoints, while the inter-frame lines are defined based on the connections of the same keypoints across consecutive frames.

GCN updates the features of the root skeleton keypoint by aggregating the local set of spatial skeleton keypoints and uses the partition strategy and residual blocks. The layer-wise propagation rule of the dual-stream GCN (2s-GCN) is initially defined as follows.

Firstly, the acquired skeleton sequence is preprocessed. The preprocessing steps for the T-frame skeleton sequence x={x_{raw}|t=1, . . . , T} involve normalization to improve stability and accelerate convergence during the training process. Specifically, the original feature vector x_{raw} of the frames consists of two sub-vectors {x_{raw,m}|m=1, 2} representing different parts of the body (upper body and head). These sub-vectors are labeled according to their partial membership, where the label variable m is set to 1 for the upper body and 2 for the head. To ensure that each sub-vector is independently normalized, the normalization process is performed on x_{raw,m}, with its mathematical representation:

$${x}_{raw , m} = \frac{{x}_{raw , m} - {\overline{x}}_{raw , m}}{σ ( {x}_{raw , m} )} ;$$

In the above formula, X̅_{raw,m} is the mean of X_{raw,m}; σ(x_{raw,m}) is the standard deviation of X_{raw,m}.

Furthermore, the partition strategy is limited to skeleton data with a complex topological structure in the spatial dimension. Specifically, Gₜ={Nₜ, Eₜ} represents the spatial graph of the skeleton at frame t, where the set of neighbors of the root skeleton keypoints vₜᵢ is specified as N(vₜᵢ)={vₜⱼ|d(vₜᵢ, vₜⱼ)≤1}. In the above formula, i and j represent skeleton keypoint labels, d(vₜᵢ, vₜⱼ) represents the minimum path length from skeleton keypoint i to skeleton keypoint j. It is important to note that different neighbor sets may vary in the number and order of skeleton keypoints, which makes direct implementation of kernel sharing infeasible. To overcome this challenge, two partition strategies are designed to divide the neighbor set into a fixed number of K subsets. A mapping function Iₜᵢ:N(vₜᵢ)→{1, . . . , K} is used to assign labels {1, . . . , K} to each skeleton keypoint vₜⱼ∈N(vₜᵢ).

A strategy for partitioning the neighbor set is based on the distance from each skeleton keypoint to a specified root skeleton keypoint, primarily used for key points in the head. This method, called distance partitioning, involves dividing the neighbor set into subgroups based on the shortest path length from each internal skeleton keypoint to the root skeleton keypoint. Formally, distance partitioning can be expressed as:

$${l}_{ti} ( {v}_{tj} ) = d ( {v}_{ti} , {v}_{tj} ) + 1 ;$$

In the above formula, Iₜᵢ(vₜⱼ) represents the label of the skeleton keypoint vₜⱼ in N(vₜᵢ). This method divides the neighbor set of a skeleton keypoint into two distinct subsets: the root skeleton keypoint and its 1-neighbor skeleton keypoints.

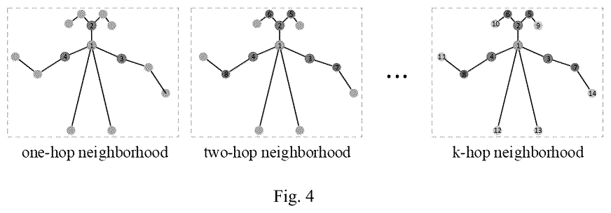

Another strategy, namely multi-scale spatial partitioning, addresses the issue of weight bias for relatively distant neighboring skeleton keypoints, as shown in FIG. 4. The self-loops in GCNs introduce more possible cycles, which can amplify bias and cause the skeletal key point sequences to be dominated by signals from local body parts. Self-loops also prevent the model from capturing the long-range key point dependencies of high-order polynomials. To address this issue, different adjacency matrices are assigned different k values to obtain different scales. This allocation method is applied to the skeletal key points of the upper body. It can be mathematically formulated as:

$${l}_{ti} ( {v}_{tj} ) = \{ \begin{matrix}1 & ifd ( {v}_{i} , {v}_{j} ) = k , \\ 1 & ifi = j \\ 0 & otherwise\end{matrix} ,$$

After classifying the skeletal data, graph convolution is performed on each part of the skeleton. The spatial aggregation strategy in graph convolution can generally be mathematically expressed as:

$${Y}_{out} ( {v}_{ti} ) = {\sum }_{{v}_{tj} ∈ N ( {v}_{ti} )} \frac{1}{{Z}_{ti} ( {v}_{tj} )} X ( {v}_{tj} ) W ( {l}_{ti} ( {v}_{tj} ) ) ;$$

In the above formula, X(vₜⱼ) represents the input features of the skeleton keypoint vₜⱼ. W(·) is a weight function, assigned from K weights based on labels lₜᵢ(vₜⱼ). Zₜᵢ(tₜⱼ) represents the number of neighbors of the skeleton keypoint vₜⱼ and normalizes the feature representation. Yₒᵤₜ(vₜᵢ) represents the output of the skeleton keypoint vₜⱼ in the graph convolution layer. Based on the partitioning strategy, the adjacency matrix A of the skeletal graph with self-loops can be divided into K matrices {Aₖ|k=1, . . . , K}. Mathematically, this can be expressed as: A=ΣₖAₖ. To illustrate, both distance partitioning and spatial configuration partitioning can be represented as A_{I}=1, where I is the identity matrix. Similarly, the degree matrix Λ can also be decomposed into K matrices {Dₖ|k=1, . . . , K} according to the same partitioning strategy. The formula for computing the graph topology structure can be expressed as:

$${Y}_{out} = σ ( {\sum }_{k = 1}^{K} {Λ}_{k}^{- \frac{1}{2}} {A}_{k} {Λ}_{k}^{- \frac{1}{2}} {XW}_{k} ) ;$$

In the above formula, σ represents the activation function.

$${Λ}_{k}^{- \frac{1}{2}} {A}_{k} {Λ}_{k}^{- \frac{1}{2}}$$

is the symmetrically normalized k-adjacency.

In the multi-scale spatial partitioning strategy, a new adjacency matrix Â is defined, leading to the following equation:

$$\hat{A} = {Λ}_{k}^{- \frac{1}{2}} ( {A}_{k} + I ) {Λ}_{k}^{- \frac{1}{2}} \\ {\hat{A}}_{k} = min ( {( {Λ}_{k}^{- \frac{1}{2}} ( {A}_{k} + I ) {Λ}_{k}^{- \frac{1}{2}} )}^{k} , 1 ) ;$$

In the above formula, min denotes the minimum function. According to the above equation, the formula for 2S-GCN on the entire input feature map is given, where N, T, and C represent the number of joints, frames, and channels, respectively.

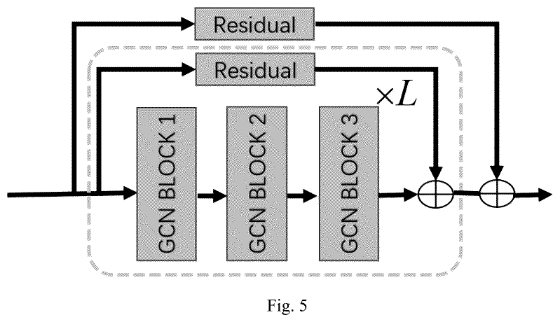

Mapping from the pose space to the feature space, then extracting pose sequence features related to upper body and head movements from the feature space. The GCN module first maps the input from the pose space to the feature space. Then, GCN blocks extract features in this feature space, with residual connections added between every three GCN blocks. This allows the network module to directly learn residuals instead of the target pose. Finally, a residual connection is added between the input pose and the output pose to ensure the network learns the differences between them. This residual connection is designed to improve the accuracy of pose feature extraction. The GCN block architecture is shown in FIG. 5.

Next, an adaptive fusion module is used to assign weights for fusing multiple features. While adding or concatenating multimodal features is common in many studies, in our task, the role of the arms is significantly more important than the secondary role of the head. Therefore, a weight allocation mechanism is designed to account for the hierarchical relationship between features.

\hat{x}_{t,m} represents a 256-dimensional feature vector obtained by the m-th part at the t-th frame of the multilayer perceptron. The formula for fusing multi-features at the t-th frame is mathematically expressed as:

$${\hat{x}}_{t} = {\sum }_{m} {α}_{m} {\hat{x}}_{t , m} ;$$

In the above formula, am represents the spatial importance weight of the m-th part assigned to the label, and is learned adaptively by the network. The

$${\sum }_{m} {α}_{m}$$

is limited to 1 and αₘ∈[0,1], and αₘ is defined as:

$${α}_{m} = \frac{exp ( {λω}_{m} )}{{\sum }_{n = 1}^{M} exp ( λ {ω}_{n} )} ;$$

In the above formula, λ is a reinforcement factor that controls the variation range of α. ω represents a set of parameters for iteratively optimizing the model, initialized to 0, and ω can be learned through standard backpropagation.

Furthermore, the information provided by frames in a skeletal sequence does not hold equal value. Key frames contain the most distinguishing information, while other frames provide contextual information. For example, in the “block stacking” action of the ASD parent-child dyad, the “hand approaching” sub-phase is considered more important than the “arms open” sub-phase. To address this issue, this disclosure designs a temporal attention module within the LSTM network to automatically assign different attention weights to different frames. The temporal attention module allows the model to more accurately capture the valuable information provided by key frames in the sequence, thereby improving model performance.

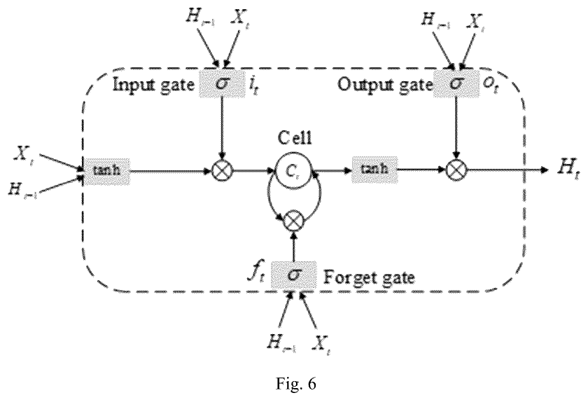

LSTM is a variant of RNN that has demonstrated exceptional ability in modeling long-term temporal dependencies in sequences. The LSTM used here consists of three gates: the input gate i_{I}, forget gate fₜ, and output gate Oₜ. These gates interact with each other to enhance the LSTM model's information analysis capability. The structure of LSTM is shown in FIG. 6.

The cell memory Cₗ exhibits temporal dynamics through its weights as recurrent edges with self-connections and interacts with the hidden state. The functionality of an LSTM cell is defined as follows:

$${i}_{t} = σ ( {W}_{xi} {X}_{t} + {W}_{hi} {H}_{t - 1} + {b}_{i} ) ; \\ {f}_{t} = σ ( {W}_{xf} {X}_{t} + {W}_{hf} {H}_{t - 1} + {b}_{f} ) ; \\ {o}_{t} = σ ( {W}_{xo} {X}_{t} + {W}_{ho} {H}_{t - 1} + {b}_{o} ) ; \\ {u}_{t} = tanh ( {W}_{xc} {X}_{t} + {W}_{hc} {H}_{t - 1} + {b}_{c} ) ; \\ {C}_{t} = {f}_{t} ▯ {C}_{t - 1} + {i}_{t} ▯ {u}_{t} ; \\ {H}_{t} = {o}_{t} ▯ tanh ( {C}_{t} ) ;$$

where □ represents the Hadamard product, σ is the sigmoid activation function, and μₜ is the modulated input.

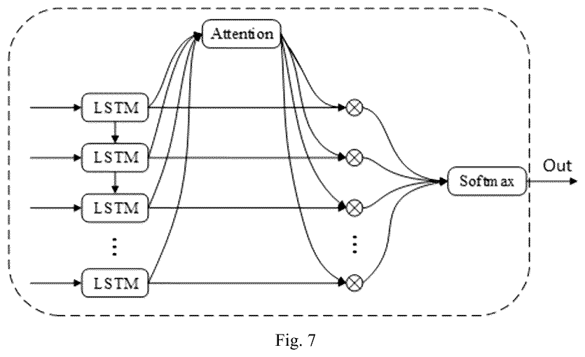

The attention layer takes the hidden states h=[hₖ, hₖ₊₁, . . . , h_{k+w-1}]^{T} as input and where hᵢ∈R^{l×m}. Based on this input, a set of attention weights αₖ, αₖ₊₁, . . . , α_{k+w-1} that represent the influence of each hidden state on the final result are computed. The model then performs a weighted sum of the inputs to obtain the result vector lₖ. The attention layer structure is shown in FIG. 7. To enhance model performance, this attention mechanism enables the model to focus more on important parts of the input sequence while paying less attention to irrelevant parts. The attention mechanism can be expressed as follows:

$${W}_{a} = W [ \begin{matrix}{x}_{t} & {h}_{t - 1}\end{matrix} ] + b \\ {e}_{i} = \frac{1}{n} {\sum }_{j} {w}_{i , j} \\ {α}_{j} = \frac{exp ( {e}_{j} )}{{\sum }_{i = k}^{k + w - 1} exp ( {e}_{j} )} \\ {l}_{k} = ReLU ( \sum {α}_{i} {h}_{i} )$$

Where W_{α}∈R^{w×n} represents the weight matrix, w_{i,j} represents the elements in the weight matrix. b∈R^{w×n} and w∈R^{w×n} are the learning parameter, and lₖ is the result vector. During model training, the model inherently learns the impact of each input element on the output and generates attention weights for each time step. As the sliding window moves, the input sequence values change, but the attention layer can dynamically compute attention weights based on input values, allowing the model to flexibly focus on variations in input values. This method helps the model capture key information in the input sequence more accurately, improving model performance and ultimately producing recognition classification results.

### Experimental Simulation

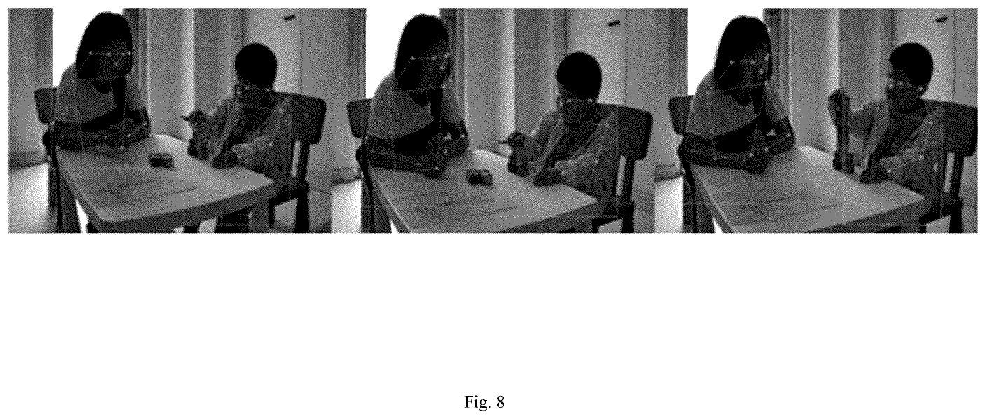

By analyzing participants' behavior during the block-building activity, it is possible to assess their ability to construct block structures under parental guidance. FIG. 8 illustrates the different interactions between children and their mothers during various time periods of block construction. The results show that children with ASD frequently require maternal assistance and struggle to meet task requirements. In contrast, typically developing (TD) children demonstrate the ability to complete block building independently. The experimental results indicate that this method achieves a high accuracy in identifying ASD patients. To ensure a reliable and objective observation of the participants' interaction abilities, standardized scenarios and structured assessments were used. In this context, upper-body and head movements were analyzed, yielding an accuracy of approximately 0.79 and an unweighted average recall (UAR) of 0.77 (Table 1).

Table 1 presents experimental results using complete data for each category. The first and second categories refer to video data of head movements and upper-body behaviors, respectively.

TABLE 1

| | | | | F [symbol] | With | Full |
|---|---|---|---|---|---|---|
| | | Class1 | Class2 | F [symbol] | Attention | data |
| ASD data | Accuracy | 0.50 | 0.68 | 0.7 [symbol] | 0.73 | 0.68 |
| | UAR | 0.57 | 0.67 | 0.69 | 0.71 | 0. [symbol] 7 |
| TD data | Accuracy | 0.59 | 0. [symbol] | 0.63 | 0.65 | 0.5 [symbol] |
| | UAR | 0.55 | 0.58 | 0. [symbol] | 0. [symbol] 3 | 0.56 |
| Full data | Accuracy | 0.57 | 0.73 | 0.7 [symbol] | 0.79 | / |
| | UAR | 0.53 | 0. [symbol] | 0.7 [symbol] | 0.77 | / |

[symbol]

indicates data missing or illegible when filed

Table 2 displays the 10-fold validation results using different physical feature selections. The worst performance occurred when using a single feature from either category 1 (head movements) or category 2 (upper-body movements). This may be due to the limited number of detected features when using only one type of movement. As shown in the “Fusion Features” column in Table 1, by using the adaptive fusion module, a maximum performance improvement of 6% (from 0.73 to 0.79) is achieved. This demonstrates the effectiveness of integrating information from multiple feature images, thereby reducing irrelevant data. As indicated in the “With Attention” column in Table 2, incorporating the attention mechanism into the model leads to an approximate 3% performance improvement.

The attention mechanism further enhances the model's ability to capture long-range frame behavior characteristics, mitigating the performance degradation typically associated with long-sequence analysis. The three rows in Table 3, labeled “ASD Data”, “TD Data” and “Complete Data” indicate that models trained using only ASD or TD children as samples performed poorly when tested on the complete dataset (which includes both ASD and TD data). This finding suggests the necessity of increasing sample and model diversity to address the limitations of models trained on single-condition data with suboptimal generalization performance.

Research results indicate that, compared to the TD children, the ASD children exhibit significant differences in block play behavior. Specifically, the ASD children demonstrate a lack of fluency and naturalness in their interactive actions, particularly when constructing block structures. Furthermore, there is a notable difference in their ability to complete block play tasks under parental guidance, with the ASD children showing less interactivity.

TABLE 2
Evaluation results of all frameworks: methods based on local descriptors,
methods based on joint posture, and methods based on CNN.

| | Accuracy | Unweighted Average Recall |
|---|---|---|
| Local Descriptor RGB-based | | |
| HOF-BOVW | 0.63 | 0.67 |
| Pose-based methods | | |
| STGCN | 0.62 | 0.64 |
| Skeleton-LSTM | 0.68 | 0.67 |
| CNN-based methods | | |
| 3DCNN | 0.44 | 0.46 |
| PoseC3D | 0.75 | 0.73 |
| Our method | | |
| 2SG-ALSTM | 0.79 | 0.78 |

In all categories, the proposed method outperforms the other methods. It is worth emphasizing that, compared to CNN-based methods, this approach achieves higher performance, because LSTM is considered superior in extracting temporal features. Overall, integrating the 2sG-ALSTM technique allows for more effective preservation of the spatiotemporal features within the dataset. The comparison between the two skeleton-based methods further confirms this point, with the proposed method achieving relatively higher accuracy than the skeleton-based LSTM approach. Notably, the 3D CNN fails to achieve better performance, which can be attributed to the inherent characteristics of the collected dataset. Unlike behavior datasets, it is difficult for CNNs to extract useful information from the background to accurately recognize behavioral activities.

### Embodiment 2

The present disclosure provides a non-transitory storage medium. The non-transitory storage medium stores a computer program; and the computer program is configured to be executed by a processor to implement the following steps.

A dataset is obtained based on a parent-child dyad block game protocol through steps of: capturing, by a camera, a facial expression and a body movement of children and a parent when the children and the parent perform a task sequence to obtain a plurality of video clips; and organizing the plurality of video clips as the dataset. The dataset is a video data; the video data is continuous; the parent-child dyad block game protocol is the task sequence.

A plurality of skeleton keypoints of a target and a position of each of the plurality of skeleton keypoints in the video data are identified based on a high-resolution network to generate a skeleton sequence. The target includes the children and the parent. The skeleton sequence includes a coordinate of each of the plurality of skeleton keypoints.

The children are classified into ASD children and TD children by inputting the skeleton sequence in a graph form into a 2sG-ALSTM network architecture to through steps of: a skeleton data in the skeleton sequence is classified into a data of an upper body of the target and a data of a head of the target. The 2sG-ALSTM network architecture is a human skeleton action recognition method based on a GCN and a LSTM network. The data of the upper body is represented as a first graph with self-loop and the data of the head is represented as a second graph with self-loop. The first graph is transformed into a first adjacency matrix and the second graph is transformed into a second adjacency matrix. The first adjacency matrix is divided into a plurality of first matrices based on a multi-scale spatial partitioning strategy. The second adjacency matrix is divided into a plurality of second matrices based on a neighbor set partitioning strategy. The plurality of first matrices are mapped from a posture space to a feature space to obtain a first vector and the plurality of second matrices are mapped from the posture space to the feature space to obtain a second vector; extracting a first posture sequence feature related to a movement of the upper body from the first vector and a second posture sequence feature related to a movement of the head from the second vector. The first posture sequence feature and second posture sequence feature are fused to obtain a first comprehensive posture sequence feature and the first comprehensive posture sequence feature is input into the LSTM network. An attention weight is automatically assigned to each frame within the first comprehensive posture sequence feature through a temporal attention module of the LSTM network to obtain a second comprehensive posture sequence feature. The children are classified into the ASD children and the TD children by predicting based on the second comprehensive posture sequence feature.

### Embodiment 3

An electronic device includes a processor, a memory and a program. The program is stored on the memory; and the processor is configured to execute the program to implement the following steps.

A dataset is obtained based on a parent-child dyad block game protocol through steps of: capturing, by a camera, a facial expression and a body movement of children and a parent when the children and the parent perform a task sequence to obtain a plurality of video clips; and organizing the plurality of video clips as the dataset. The dataset is a video data; the video data is continuous; the parent-child dyad block game protocol is the task sequence.

A plurality of skeleton keypoints of a target and a position of each of the plurality of skeleton keypoints in the video data are identified based on a high-resolution network to generate a skeleton sequence. The target includes the children and the parent. The skeleton sequence includes a coordinate of each of the plurality of skeleton keypoints.

The children are classified into ASD children and TD children by inputting the skeleton sequence in a graph form into a 2sG-ALSTM network architecture to through steps of: a skeleton data in the skeleton sequence is classified into a data of an upper body of the target and a data of a head of the target. The 2sG-ALSTM network architecture is a human skeleton action recognition method based on a GCN and a LSTM network. The data of the upper body is represented as a first graph with self-loop and the data of the head is represented as a second graph with self-loop. The first graph is transformed into a first adjacency matrix and the second graph is transformed into a second adjacency matrix. The first adjacency matrix is divided into a plurality of first matrices based on a multi-scale spatial partitioning strategy. The second adjacency matrix is divided into a plurality of second matrices based on a neighbor set partitioning strategy. The plurality of first matrices are mapped from a posture space to a feature space to obtain a first vector and the plurality of second matrices are mapped from the posture space to the feature space to obtain a second vector; extracting a first posture sequence feature related to a movement of the upper body from the first vector and a second posture sequence feature related to a movement of the head from the second vector. The first posture sequence feature and second posture sequence feature are fused to obtain a first comprehensive posture sequence feature and the first comprehensive posture sequence feature is input into the LSTM network. An attention weight is automatically assigned to each frame within the first comprehensive posture sequence feature through a temporal attention module of the LSTM network to obtain a second comprehensive posture sequence feature. The children are classified into the ASD children and the TD children by predicting based on the second comprehensive posture sequence feature.

The present disclosure is described with reference to flowcharts and/or block diagrams of methods, devices (systems), and computer program products according to embodiments of the disclosure. It should be understood that each block of the flowcharts and/or block diagrams, as well as combinations of blocks in the flowcharts and/or block diagrams, can be implemented by computer program instructions. These computer program instructions may be provided to a processor of a general-purpose computer, special-purpose computer, embedded processor, or other programmable data processing device to produce a machine, such that the instructions executed by the processor of the computer or other programmable data processing device create means for implementing the functions specified in one or more blocks of the flowcharts and/or block diagrams.

These computer program instructions may also be loaded onto a computer or other programmable data processing device to cause a series of operational steps to be performed on the computer or other programmable device to produce a computer-implemented process, such that the instructions executed on the computer or other programmable device provide steps for implementing the functions specified in one or more blocks of the flowcharts and/or block diagrams.

Although the present disclosure has been described in connection with specific embodiments and with reference to the accompanying drawings, it is not intended to limit the scope of the disclosure. Those skilled in the art will understand that various modifications or alterations can be made to the technical solutions of the present disclosure without creative effort and still fall within the scope of protection of the present disclosure.

What is claimed is:

1. A system for recognizing autism based on hybrid deep learning, comprising:
a data acquisition module;
a skeleton keypoint extraction module; and
a recognition and classification module;
wherein the data acquisition module is configured for obtaining a dataset based on a parent-child dyad block game protocol through steps of:
capturing, by a camera, a facial expression and a body movement of a child and a parent when the child and the parent perform a task sequence to obtain a plurality of video clips; and
organizing the plurality of video clips as the dataset;
wherein the dataset is a video data; the video data is continuous; the parent-child dyad block game protocol is the task sequence;
the skeleton keypoint extraction module is configured for identifying a plurality of skeleton keypoints of a target and a position of each of the plurality of skeleton keypoints in the video data based on a high-resolution network to generate a skeleton sequence; wherein the target comprises the child and the parent;
the recognition and classification module is configured for classifying the child into autism spectrum disorder (ASD) children and typically developing (TD) children by inputting the skeleton sequence in a graph form into a Two-stream Graph Attention Long Short-Term Memory (2sG-ALSTM) network architecture to through steps of:
classifying a skeleton data in the skeleton sequence into a data of an upper body of the target and a data of a head of the target; wherein the 2sG-ALSTM network architecture is a human skeleton action recognition method based on a graph convolutional network (GCN) and a long short-term memory (LSTM) network;
representing the data of the upper body as a first graph with self-loop and the data of the head as a second graph with self-loop;
transforming the first graph into a first adjacency matrix set and the second graph into a second adjacency matrix set;
selecting a first matrix from the first adjacency matrix set based on a multi-scale spatial partitioning strategy; selecting a second matrix from the second adjacency matrix set based on a neighbor set partitioning strategy;
mapping the first matrix from a posture space to a feature space to obtain a first vector and mapping the second matrix from the posture space to the feature space to obtain a second vector;
extracting a first posture sequence feature related to a movement of the upper body from the first vector and a second posture sequence feature related to a movement of the head from the second vector;
fusing the first posture sequence feature and second posture sequence feature to obtain a first comprehensive posture sequence feature; and inputting the first comprehensive posture sequence feature into the LSTM network; and
automatically assigning an attention weight to each frame within the first comprehensive posture sequence feature through a temporal attention module of the LSTM network to obtain a second comprehensive posture sequence feature;
classifying the child into the ASD children and the TD children based on the second comprehensive posture sequence feature;
wherein a route is formed by connecting the plurality of skeleton keypoints; a route distance is a distance between two of the plurality of skeleton keypoints in the route; a first adjacency matrix in the first adjacency matrix set represents a neighbor relationship between skeleton keypoints of the upper body among the plurality of skeleton keypoints corresponding to the route distance; step of selecting the first matrix from the first adjacency matrix set based on the multi-scale spatial partitioning strategy comprises:
setting a first value;
selecting the first matrix corresponding to a route distance less or equal to the first value from the first adjacency matrix set; and
wherein a second adjacency matrix in the second adjacency matrix set represents a neighbor relationship between a root skeleton keypoint and a non-root skeleton keypoint corresponding to the route distance; the root skeleton keypoint and the non-root skeleton keypoint belong to skeleton keypoints of the head among the plurality of skeleton keypoints; step of selecting the second matrix from the second adjacency matrix set based on the neighbor set partitioning strategy comprises:
setting a second value and one of the skeleton keypoints of the head as the root skeleton keypoint; and
selecting the second matrix corresponding to the route distance less or equal to the second value from the second adjacency matrix set.

2. The system of claim 1, wherein the skeleton keypoint extraction module is configured for identifying the target through steps of:
segmenting the video data into frame images;
inputting the frame images into a Faster Region-based Convolutional Neural Network (R-CNN);
extracting feature images from the frame images through a backbone network of the R-CNN;
generating human candidate regions based on the feature images through a region proposal network (RPN) of the R-CNN; and
performing classification and bounding box regression for the human candidate regions through a detection network of the R-CNN to convert the human candidate regions with varying sizes into a feature vector with fixed-size to output a coordinate of a bounding box of the target, a type of the target and a prediction probability.

3. The system of claim 2, wherein the skeleton keypoint extraction module is configured for obtaining the skeleton sequence through steps of:
inputting the coordinate of the bounding box of the target, the type of the target prediction and the prediction probability into a High-Resolution Network (HRNet);
wherein the HRNet comprises a plurality of parallel branches;
extracting space feature from the human candidate regions through the plurality of parallel branches with varying sizes of convolution kernels and varying strides to obtain multi-scale feature images; and
fusing the multi-scale feature images at both a pixel level and a channel level through a fully connected layer of the HRNet to obtain the coordinate of each of the plurality of skeleton keypoints and a confidence level of each of the plurality of skeleton keypoints to obtain the skeleton sequence.

4. The system of claim 1, wherein the first graph and the second graph are represented as G={N, E}; N represents a set of the plurality of skeleton keypoints; E represents lines connecting the plurality of skeleton keypoints.

5. The system of claim 1, wherein the GCN comprises GCN block groups; each of GCN block groups comprises three GCN blocks; the GCN block groups are connected in series; a first residual connection is set in each of the GCN block groups; a second residual connection is set between an input of a first GCN block group and an output of a last GCN block group; the recognition and classification module is configured for classifying the child into the ASD children and the TD children through steps of:
mapping the first matrix from the posture space to the feature space to obtain the first vector and mapping the second matrix from the posture space to the feature space to obtain the second vector through a first GCN block of the first block group;
extracting the first posture sequence feature from the first vector and the second posture sequence feature from the second vector by learning residuals generated by the first residual connection and the second residual connection;
performing adaptive fusion for the first posture sequence feature and the second posture sequence feature to obtain the first comprehensive posture sequence feature;
and inputting the first comprehensive posture sequence feature into the LSTM network; and
assigning the attention weight to each frame within the first comprehensive posture sequence feature through the temporal attention module of the LSTM network to obtain the second comprehensive posture sequence feature; and
predicting a probability based on the second comprehensive posture sequence feature through a softmax algorithm to classify the child into the ASD children and the TD children.

6. A non-transitory storage medium, wherein the non-transitory storage medium stores a computer program; and the computer program is configured to be executed by a processor to implement steps of
obtaining a dataset based on a parent-child dyad block game protocol through steps of:
capturing, by a camera, a facial expression and a body movement of a child and a parent when the child and the parent perform a task sequence to obtain a plurality of video clips; and
organizing the plurality of video clips as the dataset;
wherein the dataset is a video data; the video data is continuous; the parent-child dyad block game protocol is the task sequence;
identifying a plurality of skeleton keypoints of a target and a position of each of the plurality of skeleton keypoints in the video data based on a high-resolution network to generate a skeleton sequence; wherein the target comprises the child and the parent;
classifying the child into autism spectrum disorder (ASD) children and typically developing (TD) children by inputting the skeleton sequence in a graph form into a Two-stream Graph Attention Long Short-Term Memory (2sG-ALSTM) network architecture to through steps of:
classifying a skeleton data in the skeleton sequence into a data of an upper body of the target and a data of a head of the target; wherein the 2sG-ALSTM network architecture is a human skeleton action recognition method based on a graph convolutional network (GCN) and a long short-term memory (LSTM) network;
representing the data of the upper body as a first graph with self-loop and the data of the head as a second graph with self-loop;
transforming the first graph into a first adjacency matrix set and the second graph into a second adjacency matrix set;
selecting a first matrix from the first adjacency matrix set based on a multi-scale spatial partitioning strategy; selecting a second matrix from the second adjacency matrix set based on a neighbor set partitioning strategy;
mapping the first matrix from a posture space to a feature space to obtain a first vector and mapping the second matrix from the posture space to the feature space to obtain a second vector;
extracting a first posture sequence feature related to a movement of the upper body from the first vector and a second posture sequence feature related to a movement of the head from the second vector;
fusing the first posture sequence feature and second posture sequence feature to obtain a first comprehensive posture sequence feature; and inputting the first comprehensive posture sequence feature into the LSTM network; and
automatically assigning an attention weight to each frame within the first comprehensive posture sequence feature through a temporal attention module of the LSTM network to obtain a second comprehensive posture sequence feature;
classifying the child into the ASD children and the TD children based on the second comprehensive posture sequence feature;
wherein a route is formed by connecting the plurality of skeleton keypoints; a route distance is a distance between two of the plurality of skeleton keypoints in the route; a first adjacency matrix in the first adjacency matrix set represents a neighbor relationship between skeleton keypoints of the upper body among the plurality of skeleton keypoints corresponding to the route distance; step of selecting the first matrix from the first adjacency matrix set based on the multi-scale spatial partitioning strategy comprises:
setting a first value;
selecting the first matrix corresponding to a route distance less or equal to the first value from the first adjacency matrix set; and
wherein a second adjacency matrix in the second adjacency matrix set represents a neighbor relationship between a root skeleton keypoint and a non-root skeleton keypoint corresponding to the route distance; the root skeleton keypoint and the non-root skeleton keypoint belong to skeleton keypoints of the head among the plurality of skeleton keypoints; step of selecting the second matrix from the second adjacency matrix set based on the neighbor set partitioning strategy comprises:
setting a second value and one of the skeleton keypoints of the head as the root skeleton keypoint; and
selecting the second matrix corresponding to the route distance less or equal to the second value from the second adjacency matrix set.

7. An electronic device, comprising:
a processor;
a memory; and
a program;
wherein the program is stored in the memory; and the processor is configured to execute the program to implement steps of:
obtaining a dataset based on a parent-child dyad block game protocol through steps of:
capturing, by a camera, a facial expression and a body movement of a child and a parent when the child and the parent perform a task sequence to obtain a plurality of video clips; and
organizing the plurality of video clips as the dataset;
wherein the dataset is a video data; the video data is continuous; the parent-child dyad block game protocol is the task sequence;
identifying a plurality of skeleton keypoints of a target and a position of each of the plurality of skeleton keypoints in the video data based on a high-resolution network to generate a skeleton sequence; wherein the target comprises the child and the parent;
classifying the child into autism spectrum disorder (ASD) children and typically developing (TD) children by inputting the skeleton sequence in a graph form into a Two-stream Graph Attention Long Short-Term Memory (2sG-ALSTM) network architecture to through steps of:
classifying a skeleton data in the skeleton sequence into a data of an upper body of the target and a data of a head of the target; wherein the 2sG-ALSTM network architecture is a human skeleton action recognition method based on a graph convolutional network (GCN) and a long short-term memory (LSTM) network;
representing the data of the upper body as a first graph with self-loop and the data of the head as a second graph with self-loop;
transforming the first graph into a first adjacency matrix set and the second graph into a second adjacency matrix set;
selecting a first matrix from the first adjacency matrix set based on a multi-scale spatial partitioning strategy; selecting a second matrix from the second adjacency matrix set based on a neighbor set partitioning strategy;
mapping the first matrix from a posture space to a feature space to obtain a first vector and mapping the second matrix from the posture space to the feature space to obtain a second vector;
extracting a first posture sequence feature related to a movement of the upper body from the first vector and a second posture sequence feature related to a movement of the head from the second vector;
fusing the first posture sequence feature and second posture sequence feature to obtain a first comprehensive posture sequence feature; and inputting the first comprehensive posture sequence feature into the LSTM network; and
automatically assigning an attention weight to each frame within the first comprehensive posture sequence feature through a temporal attention module of the LSTM network to obtain a second comprehensive posture sequence feature;
classifying the child into the ASD children and the TD children based on the second comprehensive posture sequence feature;
wherein a route is formed by connecting the plurality of skeleton keypoints; a route distance is a distance between two of the plurality of skeleton keypoints in the route; a first adjacency matrix in the first adjacency matrix set represents a neighbor relationship between skeleton keypoints of the upper body among the plurality of skeleton keypoints corresponding to the route distance; step of selecting the first matrix from the first adjacency matrix set based on the multi-scale spatial partitioning strategy comprises:
setting a first value;
selecting the first matrix corresponding to a route distance less or equal to the first value from the first adjacency matrix set; and
wherein a second adjacency matrix in the second adjacency matrix set represents a neighbor relationship between a root skeleton keypoint and a non-root skeleton keypoint corresponding to the route distance; the root skeleton keypoint and the non-root skeleton keypoint belong to skeleton keypoints of the head among the plurality of skeleton keypoints; step of selecting the second matrix from the second adjacency matrix set based on the neighbor set partitioning strategy comprises:
setting a second value and one of the skeleton keypoints of the head as the root skeleton keypoint; and
selecting the second matrix corresponding to the route distance less or equal to the second value from the second adjacency matrix set.
